(19) United States
(12) Patent Application Publication
(10) Pub. No.: US 2026/0234523 A1
(43) Pub. Date: Aug. 13, 2026

(54) SYSTEMS, DEVICES, AND PROCESSES FOR MINIATURIZED PHYSIOLOGICAL MIMICRY

(71) Applicant: Research Institute at Nationwide Children’s Hospital, Columbus, OH (US)

(72) Inventors: Belgacem Mihi, Columbus, OH (US); Gail E. Besner, Columbus, OH (US)

(21) Appl. No.: 19/160,443

(22) PCT Filed: Feb. 29, 2024
(86) PCT No.: PCT/US2024/017896

§ 371 Date: Aug. 27, 2025

Related U.S. Application Data
(60) Provisional application No. 63/449,134, filed on Mar. 1, 2023.

(51) Int. Cl. C12M 3/06; B29C 33/38; B29C 33/40; B29C 39/02; B29C 39/26; B29L 31/00; B33Y 80/00; C12M 1/00; C12M 1/12; C12M 3/00

(52) U.S. Cl. CPC C12M 23/16; B29C 33/3842; B29C 33/40; B29C 39/02; B29C 39/26; B33Y 80/00; C12M 21/08; C12M 25/14; C12M 29/10; B29K 2089/00; B29L 2031/756

(57) ABSTRACT

A miniaturized physiological mimicry platform is disclosed. The platform may include microfluidic chips and other microphysiological devices and their use to emulate the anatomies and/or functions of tissues, such as single and multiple organ systems. Various manufacturing approaches are disclosed for fabricating chips of different sizes. Formulations of extracellular matrices that better sustain attachment and expansion of different cell types various devices are also provided.

### CROSS-REFERENCE TO RELATED APPLICATIONS

This application claims priority to and the benefit of U.S. Provisional Patent Application No. 63/449,134 filed Mar. 1, 2023, the entirety of which is incorporated herein by reference.

### BACKGROUND

Drug discovery, or translating compounds into effective drugs, has traditionally been slow due to the paucity of available models. Cell cultures fail to effectively model tissues and interactions between tissues because they lack the fluidic movements found in organisms. Animal models such as rodents have significant differences in genetics and physiology, and effects of a compound being tested in one organ of an animal does not apply to the whole organism with high fidelity. Current approaches to emulating cellular function are costly to produce and can only incorporate a limited number of cell types, limiting their ability to functionally recapitulate human native tissues. Existing devices are mainly made with biologically inert polydimethylsiloxane (PDMS) using a soft lithography or molding technique, which is very costly.

### SUMMARY

This disclosure relates to miniaturized physiological mimicry devices, systems, and processes, such as microfluidic chips and other microphysiological devices and their use to emulate the anatomies and/or functions of tissues, such as single and multiple organ systems. This disclosure additionally relates to processes for manufacturing such systems and devices, including methods of microfluidic organ-on-a-chip (e.g., a single organ on one chip) or organs-on-chip (e.g., multiple organs on a chip) fabrication that may, in some embodiments, employ three-dimensional (3D) printing techniques. This disclosure moreover relates to formulations of extracellular matrices that better sustain attachment and expansion of different cell types in various devices. Organ-on-a-chip and organs-on-a-chip are used interchangeably with tissue-on-a-chip and tissues-on-a-chip, respectively. A device that includes multiple chips (e.g., multiple organ-on-chips and/or multiple organs-on-chips) may, for convenience, be referred to herein as simply organ-on-a-chip and/or organs-on-a-chip. An organ(s)-on-a-chip/tissue(s)-on-a-chip is a device that may include mechanical, electrical, and/or biological components and ingredients to model the functions and/or structures of organs and/or other tissues, often but not necessarily on a scale that is smaller than the organs and/or other tissues being modeled.

In one aspect, various embodiments relate to a microfluidic device for modeling one or more physiological functions of one or more organisms, the microfluidic device comprising a first channel, and a second channel separated from the first channel by an extracellular matrix (ECM) that mimics a connective tissue of the organism. Example physiological functions that can be modeled include digestive functions, respiratory functions, hepatic functions, urinary functions, and/or other functions. Example connective tissues include tissues of the intestine, lung, liver, and/or other tissues. In various embodiments, an organism is a multicellular organism, such as a human or non-human animal.

In various embodiments, the ECM comprises a first layer at least partly positioned on a first outer side of the first channel, and a second layer at least partly positioned on a second outer side of the second channel. In various embodiments, the first layer covers between 50% and 100% of the first channel, such as at least 50%, at least 55%, at least 60%, at least 65%, at least 70%, at least 75%, at least 80%, at least 85%, at least 90%, at least 95%, or at least 99% of the first channel. In various embodiments, the second layer covers between 50% and 100% of the second channel, such as at least 50%, at least 55%, at least 60%, at least 65%, at least 70%, at least 75%, at least 80%, at least 85%, at least 90%, at least 95%, or at least 99% of the second channel.

In various embodiments, the ECM is part of a lamina propria of a selected tissue of an organism, where the lamina propria is composed of ECM and cells. In example embodiments, the ECM can be isolated from animal tissue or cell lines. In certain embodiments, synthetic ECM may be used.

In various embodiments, the first channel is in fluid communication with a first set of one or more reservoirs, and the second channel is in fluid communication with a second set of one or more reservoirs.

In various embodiments, the microfluidic device is an organ-on-a-chip that simulates one or more physiological functions of an organ.

In various embodiments, the microfluidic device further comprises a set of one or more additional channels for modeling interaction between at least two organs or other tissues. In various embodiments, example interactions include use of gut and brain in a chip to emulate a gut-brain axis, mimicking interaction between gut and liver to study enterohepatic recirculation of drugs, and/or emulating interactions between the brain and the circulatory system to study the blood-brain barrier.

In various embodiments, the ECM comprises any combination of basement membrane matrix, one or more proteins and glycoproteins such as such as collagens, elastin, laminins and tenascins, proteoglycans and glycosaminoglycans, hyaluronan, polyethylene glycol n-hydroxysuccinimide (PEG-NHS), and/or one or more salts.

In various embodiments, the ECM comprises: one or more insoluble proteins; collagen; a basement membrane matrix; polyethylene glycol n-hydroxysuccinimide (PEG-NHS); one or more glycoproteins; and a salt.

In various embodiments, wherein the one or more insoluble proteins are, or comprise, fibrin. In various embodiments, the collagen is, or comprises, type I collagen. In various embodiments, the one or more glycoproteins are, or comprise, laminin.

In various embodiments, the ECM extends over both the first channel and the second channel.

In various embodiments, the ECM is, or comprises, a first layer comprising epithelial cells and a second layer comprising endothelial cells.

In another aspect, various embodiments relate to a method of fabricating a microfluidic device, the method comprising: using a sacrificial material to three-dimensional (3D) print a set of channels and a set of reservoirs; covering the set of channels with an extracellular matrix (ECM); and warming the sacrificial material to yield perfusable channels once the set of channels is flushed. In example embodiments, the sacrificial material is gelatin that is liquified by warming the gelatin at 37 degrees Celsius.

In various embodiments, the method further comprises rocking the microfluidic device to allow media to go between reservoirs through the set of channels, wherein the rocking provides perfusion of the set of channels.

In various embodiments, the ECM is part of a lamina propria of an organ or tissue of an organism.

In various embodiments, the warming is performed after embedding the set of channels and the lamina propria in a fibrin-PEG-NHS gel.

In various embodiments, the sacrificial material is, or comprises, a sacrificial ink.

In various embodiments, the sacrificial ink is, or comprises, F127. In various embodiments, the F127 is liquefied by warming the microfluidic device.

In various embodiments, the sacrificial material is, or comprises, a gelatin. In various embodiments, the gelatin is liquefied at about 37 degree Celsius.

In another aspect, various embodiments relate to a method of fabricating a microfluidic device, the method comprising: printing a set of channels and a set of reservoirs; casting a sacrificial material into the set of channels; embedding the set of channels in an extracellular matrix; and flushing the sacrificial material from the set of channels.

In various embodiments, the sacrificial material is, or comprises, a gelatin. In various embodiments, the method further comprises incubating the microfluidic device to liquefy the gelatin (at, e.g., about 37 Celsius).

In various embodiments, the set of channels are micromesh channels printed with a photocurable polymer resin. Various embodiments relate to and/or include a method or process that comprises steps taken to manufacture or develop the devices disclosed herein, such as a step of printing a set of channels (e.g., micromesh channels) with a photocurable polymer resin.

In various embodiments, wherein the set of channels are patterned by casting liquid gelatin into a printed resin mold.

In various embodiments, the method further comprises obtaining, through gelation of the resin mold, a gelatin structure that is successively encapsulated in the ECM.

In various embodiments, the method further comprises, after gelation of the ECM, melting and flushing the gelatin structure to leave behind the set of channels separated by the ECM and connected to the set of reservoirs.

In various embodiments, the set of channels are printed using a digital light processing (DLP) printer or a stereolithography (SLA) printer.

In various embodiments, the set of channels are micromesh channels printed with a photocurable polymer resin.

Various embodiments relate to other systems, devices, and methods disclosed herein, such as chips or other microfluidic devices manufactured according to the disclosed fabrication processes.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1A depicts an example two-channel microfluidic device according to various example embodiments. FIG. 1B is an image of an example device according to various embodiments.

FIG. 2 depicts an example multi-channel microfluidic device according to various example embodiments.

FIG. 3 depicts another example microfluidic device according to various example embodiments.

FIG. 4 depicts another example microfluidic device according to various example embodiments.

FIG. 5 depicts another example microfluidic device according to various example embodiments.

FIG. 6 depicts another example microfluidic device according to various example embodiments.

FIG. 7 illustrates an example multi-organ microfluidic device according to various example embodiments

FIG. 8 depicts a process for developing microfluidic devices according to various example embodiments.

FIG. 9 depicts another process for developing microfluidic devices according to various example embodiments.

FIGS. 10A and 10B depict microscopic images of channels separated by a matrix according to various example embodiments.

FIGS. 11A and 11B depict microscopic images of channels corresponding to FIGS. 10A and 10B, respectively, according to various example embodiments.

FIG. 12 depicts another process for developing microfluidic devices according to various example embodiments.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior disclosure.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

As used herein, the term “optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1 %.

The term “substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As utilized herein, the terms “approximately,” “about,” “substantially”, “essentially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims. In certain example embodiments, the terms “approximately,” “about,” “substantially”, “essentially,” and similar terms can indicate that a specified value can vary up to ten percent (e.g., value +/−10%).

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

F 127™ (also known as Pluronic F-127™ (poloxamer 407) or Pluronic F-127 20% to 40% wt/v) is an injectable synthetic hydrogel that has a reversible mechanism for gelation and is nontoxic, biocompatible, and biodegradable. In example embodiments, Pluronic F-127™ at concentrations ranging between 10 and 20% may be used. It has been used as a sacrificial bioink for temporary support or to create channels, vessels or vasculature. It is commercially available from a variety of vendors, for example Allevi (see https://www.allevi3d.com/product/pluronic/), Advanced BioMatrix (see https://advancedbiomatrix. com/pluronic-40-sterile-solution. html) and ThermoFischer (see https://www. thermofisher. com/order/catalog/product/P3000MP).

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting.

Other embodiments may be utilized, and other changes may be made, without departing from the spirit or scope of the subject matter presented here. It will be readily understood that the aspects of the present disclosure, as generally described herein, and illustrated in the figures, may be arranged, substituted, combined, and designed in a wide variety of different configurations, all of which are explicitly contemplated and make part of this disclosure.

### Modes for Carrying Out the Disclosure

Despite extraordinary technological advances, investigational drug discovery remains inefficient, with 85% of therapies failing in early clinical trials, and only half of those selected for phase III trials approved for human use. The poor success rates of translation from bench to bedside are mainly attributed to deficient preclinical models, thus there is an urgent need for novel technologies to model human disease. Organ-on-a-chip platforms can thus provide reliable alternatives to animal testing in preclinical settings, and can be used as a predictive tool for personalized precision medicine. Organ-on-a-chip devices, in contrast to growing cells under static or non-moving conditions, provide a fluidic moving component on the chip.

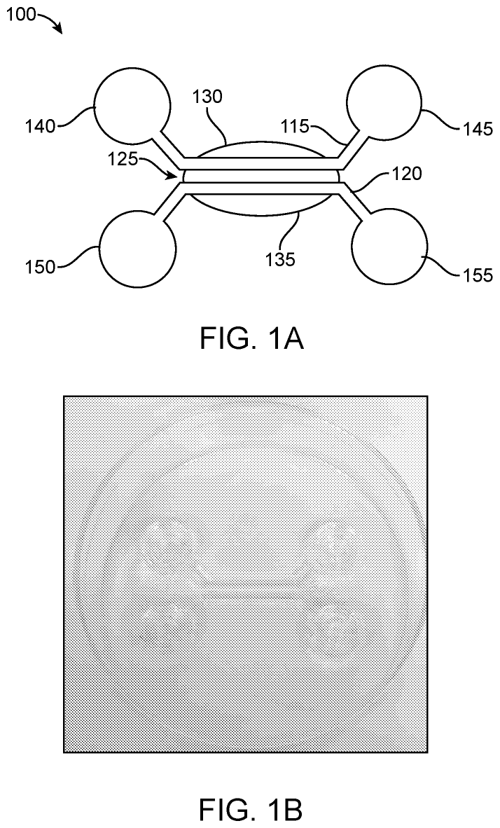

FIG. 1 provides a top view of an improved organ-on-a-chip system 100 according to various potential embodiments. Organ-on-a-chip 100 includes a set of two channels (though other versions may have other numbers of channels, such as more than two), which in the example depicted in FIG. 1 includes a first channel 115 and a second channel 120 that is separated from the first channel 115 by an extracellular matrix (ECM) 125. In various embodiments, the ECM lines the channels without penetrating the channels. Channels may mimic, for example, a vasculature of an organism (or other paths of fluid flow). The extracellular matrix 125 may mimic a connective tissue of an organism. The extracellular matrix 125 may include a first layer 130 and a second layer 135. The first layer 130 may at least partly be located on a first outer side of the first channel 115 (“above” the first channel 115 as organ-on-a-chip 100 is oriented in FIG. 1) and the second layer 135 may at least partly be located on a second outer side of the second channel 120 (“below” the second channel 120 as organ-on-a-chip 100 is oriented in FIG. 1). The first channel 115 is connected to a first set of one or more reservoirs (e.g., reservoirs 140 and 145 in FIG. 1), and the second channel 120 is connected to a second set of one or more reservoirs (e.g., reservoirs 150 and 155 in FIG. 1). The first and second sets of reservoirs may be, for example, culture medium reservoirs. The first set of reservoirs in FIG. 1 includes reservoirs 140 and 145, and the second set of reservoirs in FIG. 1 includes reservoirs 150 and 155.

In a non-limiting example, the organ-on-a-chip may be used to model, for example, the intestine (a sort of “gut-on-chip”). The intestine is a complex organ composed of multiple layers containing multiple different cell types. The inner lining is the mucosa, which has finger-like projections that extend towards the lumen called villi, and deep crypts in-between the villi. The lamina propria of the mucosa is deep to the epithelial layer and contains blood vessels and immune cells. The mucosa is surrounded by layers of muscle containing the nerves that are responsible for intestinal motility. In example intestinal organ-on-a-chips, the extracellular matrix may comprise, for example, myofibroblasts, the first layer may be or may comprise intestinal epithelial cells, and the second layer may be or may comprise intestinal endothelial cells.

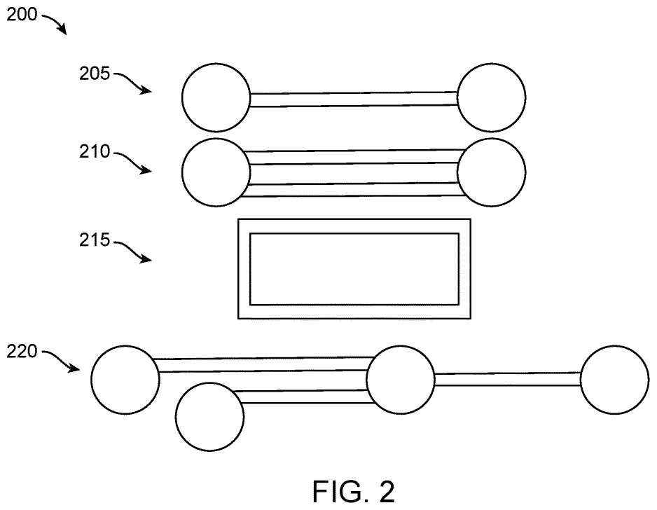

FIG. 2 illustrates the versatility and potential structural variation in other example implementations. For example: a device can have one channel (e.g., 205) or more than two channels (e.g., 220); channels need not have reservoirs (e.g., 215); channels need not be linear, and can have other shapes (e.g., square as depicted at 215); channels can form closed loops in which opposing ends connect with each other (e.g., 215); a reservoir can feed into one channel (e.g., 205) or multiple channels (e.g., 210, 220); one channel does not necessarily feed into two reservoirs; etc. Such variation is well-suited to, for example, mimicking the structure and function of various different multi-organ system; simulating the introduction of different bioactive components from different sources and at different times; controlling the flow of fluids to more precisely control what agents or media (or combinations of agents and/or media) come into contact with other agents and/or media (or other combinations of agents and/or media) and with other cells. The variations may be employed to simulate the structure of, functionality of, and interactions between and among any number of organs or other tissues.

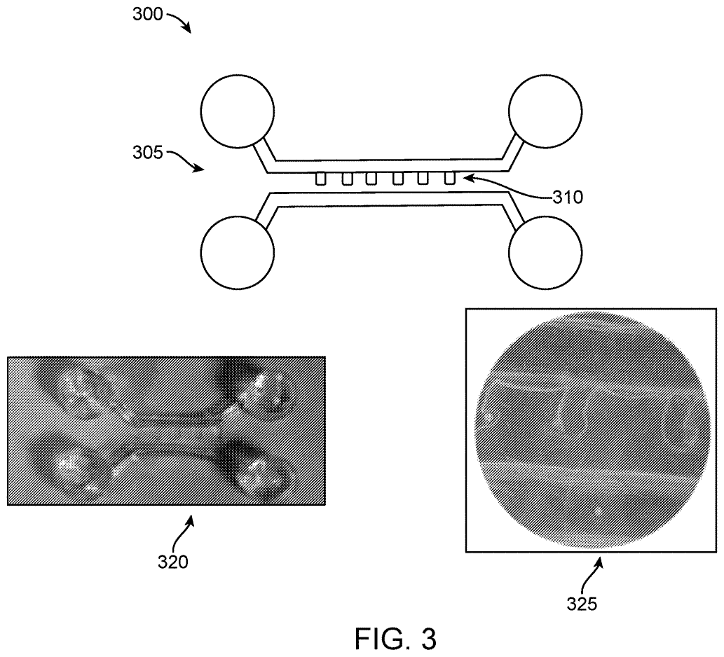

In other embodiments, such as the example device 300 depicted in FIG. 3, any of the channels may have protrusions 310 that extend at least partly into a region (e.g., an ECM between channels) and towards another channel or other structure. Such protrusions 310 can be used to simulate, for example, various different physiological structures and functions (e.g., intestinal villi or chorionic villi), and can be incorporated to, for example, increase surface area and/or facilitate various processes such as absorption of nutrients and/or cellular communication. At 320, FIG. 3 provides an image of an example device with protrusions, and at 325, FIG. 3 provides a zoomed-in view of example protrusions.

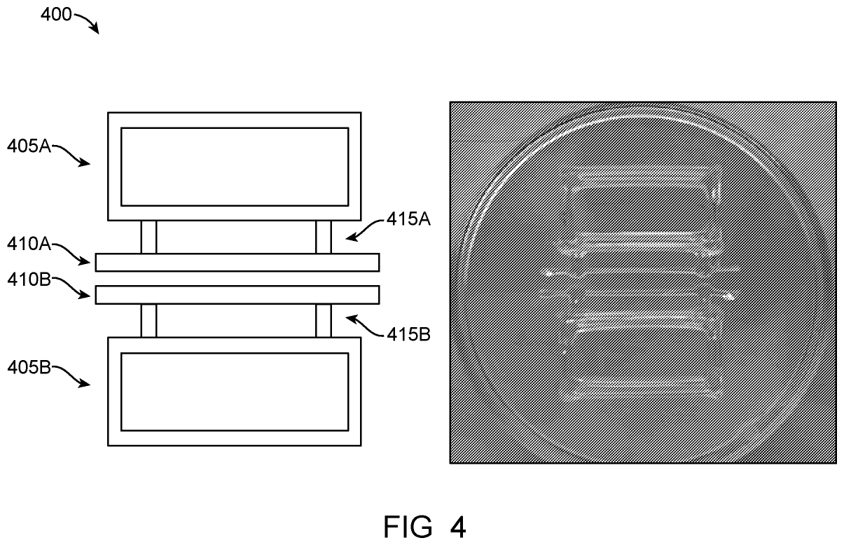

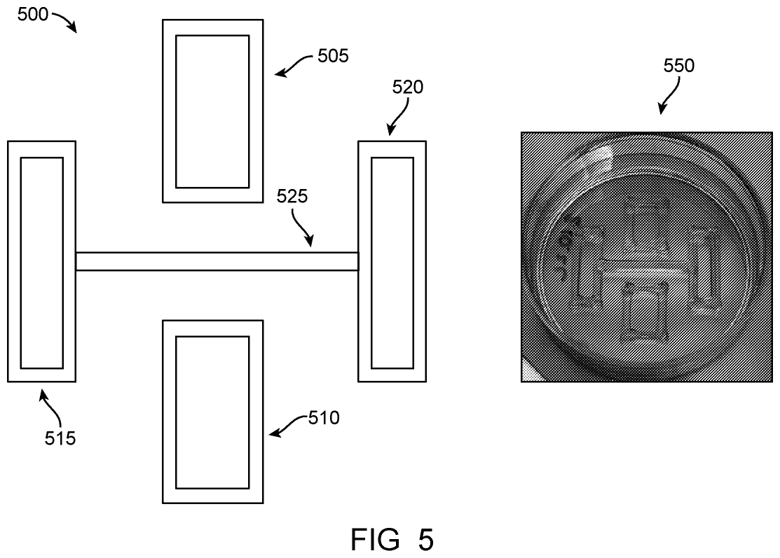

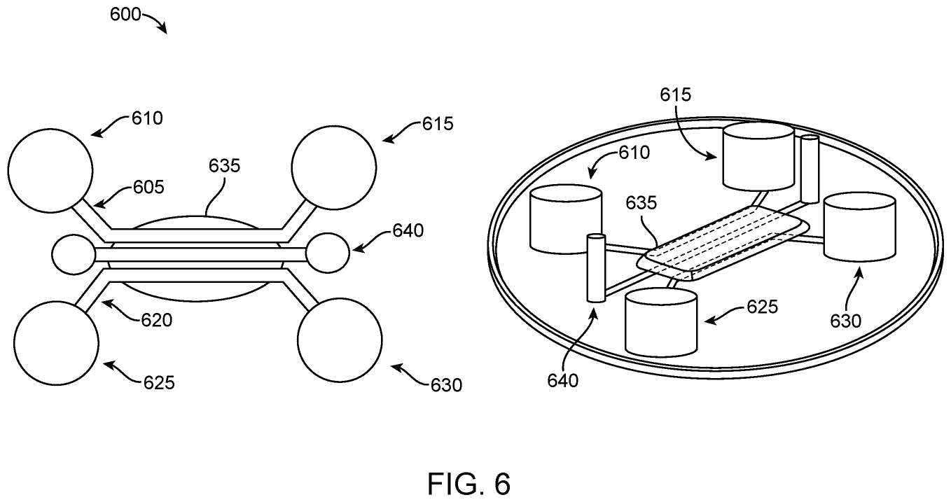

FIG. 4 depicts another example of a multi-channel device 400 with two rectangular structures 405A and 405B, each rectangular structure connected to a respective primary channel 410A and 410B via respective secondary channel 415A and 415B. FIG. 5 depicts an example device 500 that includes rectangles 505, 510, 515, and 520, with rectangles 515 and 520 connected by channel 525. At 550, FIG. 5 provides an image of such an example device. FIG. 6 depicts an example device 600, with a top view on the left and a perspective view on the right. Device 600 includes a first channel 605 extending between reservoirs 610 and 615, and a second channel 620 extending between reservoirs 625 and 630. An ECM 635 extends across the first and second channels 605 and 625. A pillar 640 may be included to serve as a cell loading channel for the ECM 635.

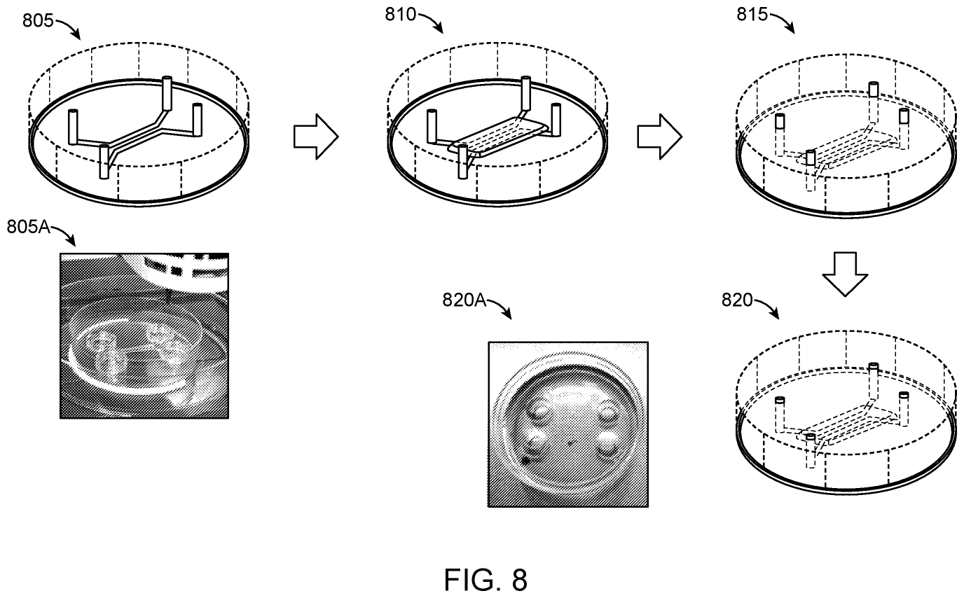

In various embodiments, the various implementations of organ-on-a-chips may be manufactured in different ways depending on desired configurations. Referring to FIG. 8, in various embodiments, at 805, the sets of channels and reservoirs may be printed using a sacrificial ink, such as F127. An image of an example component resulting from step 805 is provided at 805A. At 810, the two channels may be covered with a thin layer of extracellular matrix (ECM), which may be a part of a lamina propria or other lining (e.g. mucosae) of select tissues in the body. Other components may be incorporated into the extracellular matrix, such as immune cells and enteric nervous system cells. In various embodiments, the extracellular matrix may be composed of fibrin or other insoluble protein(s), collagen (e.g., Type I collagen), a basement membrane matrix or attachment substrate (e.g., Matrigel), polyethylene glycol n-hydroxysuccinimide (PEG-NHS), laminin (or other glycoproteins), and a suitable salt component (e.g., 250 mM NaCl). A prior coating of the plate (used interchangeably with “dish”) with polydopamine (20 mg/ml) prior to printing the channels and the reservoirs can significantly enhance adhesion of the extracellular matrix to the plate. The dish/plate may be a polystyrene dish/plate used to generate the chip. The dish/plate need not be a polystyrene dish, as any type of biocompatible clear plastic or resin dish/plate can be used in the fabrication process. After embedding the channels and the lamina propria in a fibrin-PEG-NHS gel at 815, the organ-on-a-chip may be warmed to liquefy the sacrificial ink at 820, which yields perfusable channels once flushed. An image of an example component resulting from step 820 is depicted at 820A. The channels are connected to reservoirs that contain cell culture medium.

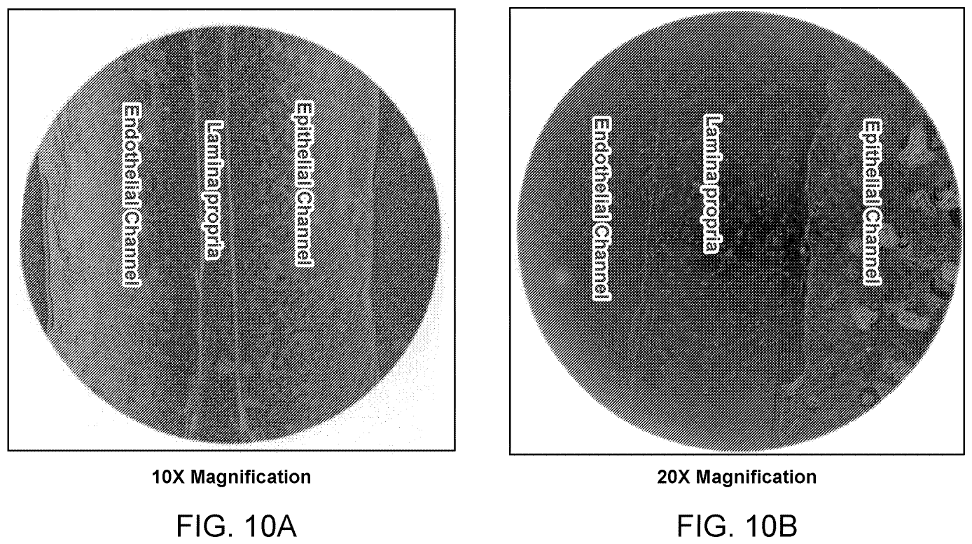

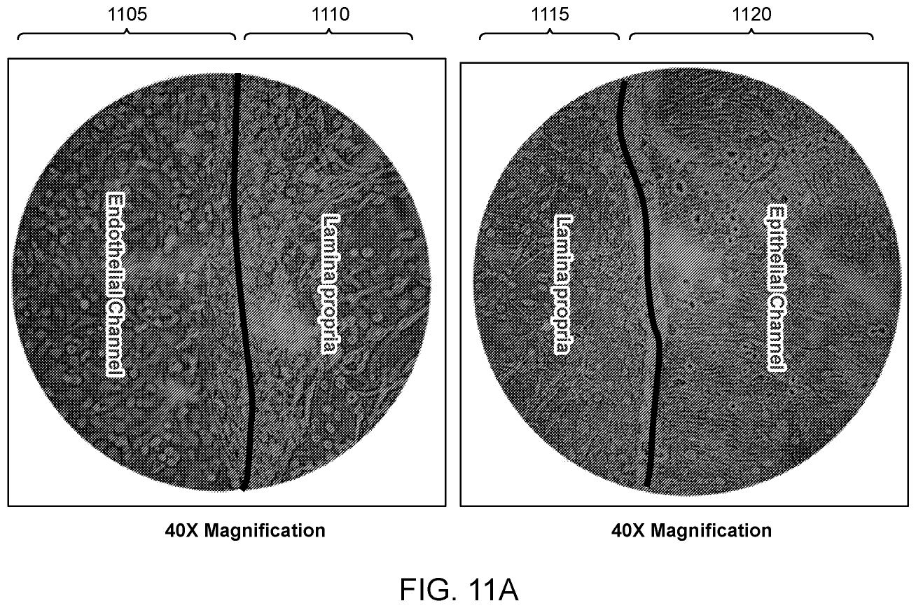

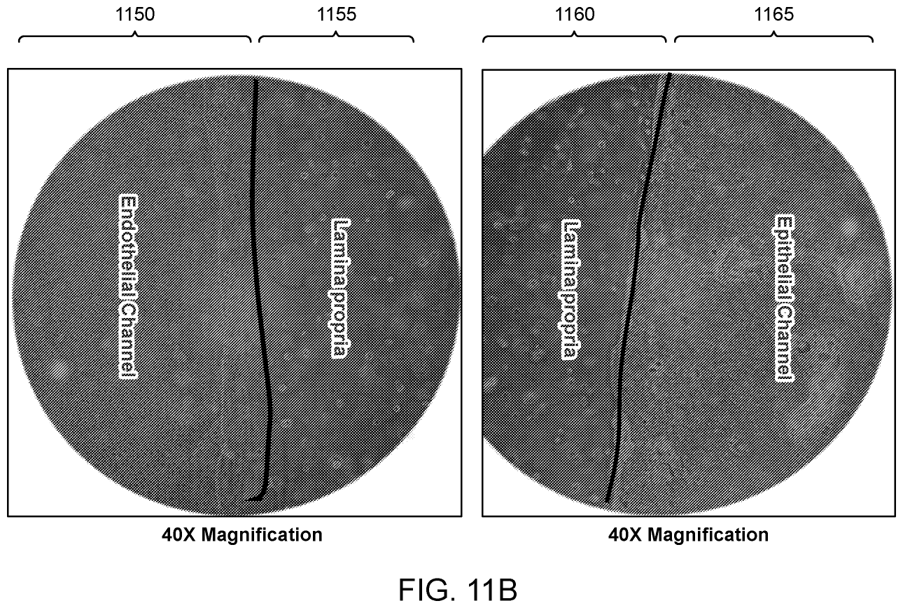

As an example, and with reference to FIGS. 10A and 11A, to produce a gut-on-chip, a first channel (1120) may be seeded with human primary intestinal epithelial cells harvested from a patient, while as second channel (1105) may be seeded with commercially available human primary endothelial cells. The reservoirs may then be filled with cell culture medium. The cells may be initially cultured under static conditions for, for example, 24 to 48 hours.

Cell maturation may be achieved via channel perfusion by rocking the organ-on-a-chip in an orbital shaker for, for example, 7 to 12 days. By rocking the plate, the culture medium travels from one medium reservoir to another medium reservoir, creating a flow through the connecting channel. The shear stress generated by the movement of the culture media contributes to the maturation of the cells in the channel. The speed of the rocker depends on the desired shear stress and the perfusion. Epithelial and endothelial cells expand in the channel to form cell monolayers separated by the lamina propria (1110 and 1115) which contains the intestinal myofibroblasts. As used herein, static conditions refers to a culture when there is no perfusion of the channel (e.g., no movement of the culture media in the channel). The chip may be left in the incubator without changes or manipulations (e.g., not on a rocker). By contrast, when the chip is placed on a rocker, a liquid flow is generated in the channel, as a result of the medium going from one reservoir to another reservoir through the connecting channel.

In various embodiments, given the critical importance of compatibility with a rapid high-throughput screening of drug candidates, reduction of the size of the platform is an important consideration. The use of F127 sacrificial ink may be suitable for generation of, for example, a 5 centimeter (cm) gut-on-chip. For chip sizes smaller than about 3 cm, alternative approaches may be used, as discussed below.

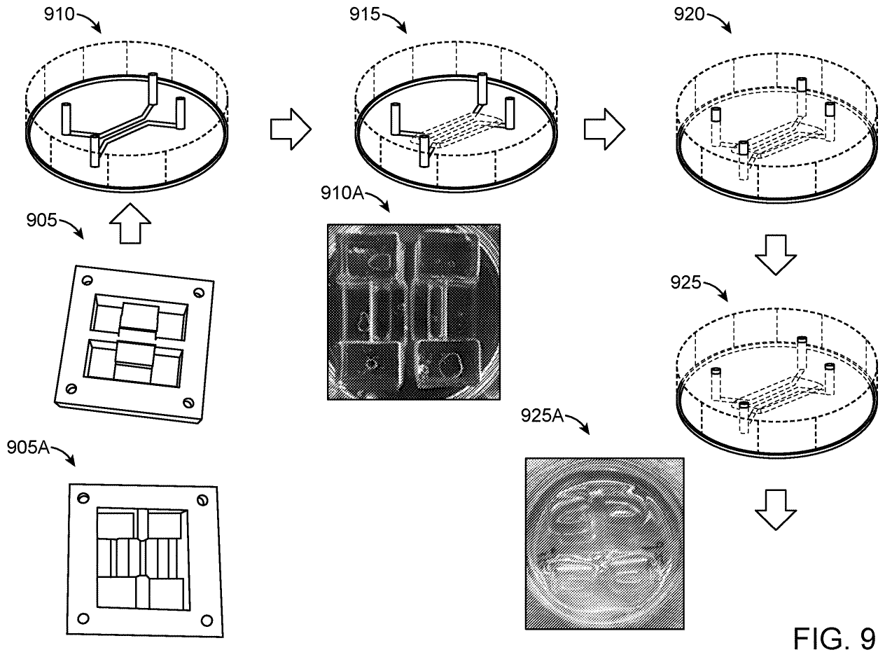

In various alternative embodiments, and with reference to FIG. 9, a gelatin may be used as a sacrificial material (instead of F127). Two channels connected to culture medium reservoirs may be patterned by casting liquid that is, for example, between 7.5% to 20% gelatin inclusive (e.g., 15% gelatin) into a printed resin mold (905, an image of an example of which is provided at 905A). After gelation at 4 degrees Celsius (C), the resulting gelatin structure (910, an image of an example of which is provided at 910A) may be successively encapsulated in the extracellular matrix (915) containing, for example, myofibroblasts or other suitable cells depending on the tissue functionality being simulated. For example, the resulting gelatin structure (including the channels and the reservoirs) may first be placed on a thin layer of the ECM. The channels may then be covered as with the ECM containing the myofibroblasts or other suitable cells. After the gelation of the extracellular matrix (920), the gelatin may be incubated/melted at 37 degrees C, and flushed (925), leaving behind two channels separated by the extracellular matrix and connected to culture medium reservoirs (see, e.g., 925A). This approach may be suitable for, for example, generating 2 cm chips.

This fabrication approach does not negatively affect the attachment of epithelial cells to the channels, as can be seen in FIGS. 10B and 11B (which depicts an endothelial channel 1150, lamina propria 1155 and 1160, and an epithelial channel 1165). Epithelial and endothelial cells expand in the channel to form cell monolayers separated by the lamina propria which contains the myofibroblasts.

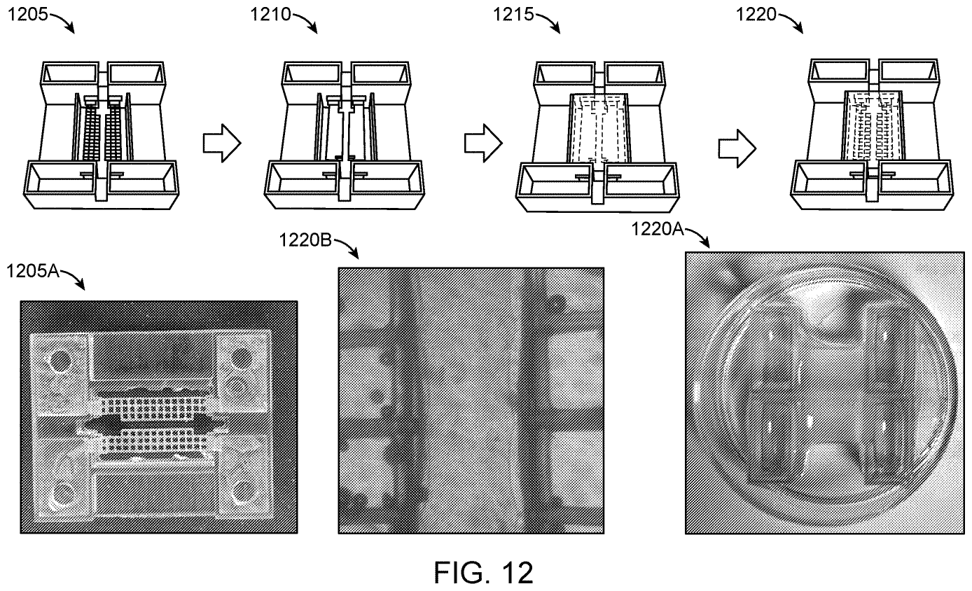

In various other embodiments, and with reference to FIG. 12, to further miniaturize the platform and enhance its complexity, a microfluidic system that uses digital light processing/stereolithography (DLP/SLA) 3D printing has been developed. A chip can be printed with a DLP or SLA 3D printer (both of which may use photocurable material). A 3D file of the chip may be generated using 3D software (such as Blender). After uploading the file into the printer, the chip may be printed with a biocompatible material (1205, an image of an example of which is provided at 1205A). The resulting chip is cleaned in an alcohol, cured under ultraviolet (UV) light, and subjected to plasma treatment.

The chip comprises media reservoirs connected by micromesh channels made of photocurable polymer resin. Due to the surface tension between the liquid and the solid surface of the micromesh structure, the micromesh structure can trap a liquid and behave as a microfluidic channel. The micromesh channels may be filled with 15% liquid gelatin (1210), which can be allowed to gelify at 4 degrees C. The channels may be covered with an extracellular matrix (1215) containing, for example, human myofibroblasts or other cells.

After the gelation of the extracellular matrix, the gelatin may be incubated/melted at 37 degrees C, and flushed (1220), yielding two perfusable channels separated by the extracellular matrix (see, e.g., 1220A and 1220B). This approach allows for the reduction of the size of the chip to less than about 1 cm. The perfusion of the microchannels (which are separated with an extracellular matrix) may be achieved by rocking the chip, allowing the media to go from a reservoir to another through the connecting channels.

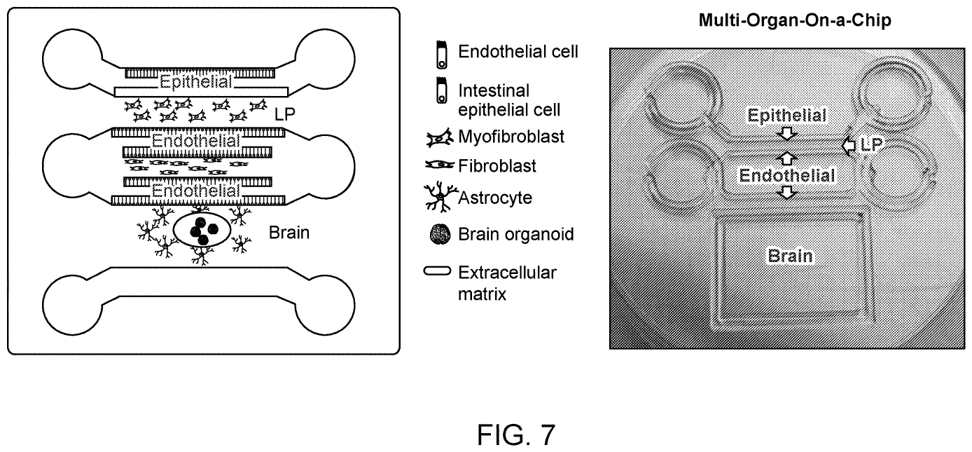

In various embodiments, given the complexity of interactions between different organs in animal (e.g., human) pathologies, a multi-organ-on-a-chip can provide better mimicry of the events taking place in the animal body (e.g., human body or body of another organism) during disease. Higher complexity can be achieved by integrating different organs or other tissues in a single chip to produce, for example, a “gut-brain” chip (see, e.g., FIG. 7), or another “Tissue 1-Tissue 2” chip, where Tissue 1 and Tissue 2 can be selected from any tissues or cells of interest, such as brain, lung(s) liver, kidney(s), heart, bladder, stomach, intestines, skin, pancreas, spleen, thyroid, joints, immune system, etc. In various embodiments, chips need not be limited to the study of the interaction of two tissues and may be multi-tissue chips (e.g., “Tissue 1-Tissue 2-Tissue 3” chips, or otherwise “Tissue 1 Tissue 2- . . . -Tissue n” chips, where n is the number of relevant tissues of interest with a structure or function represented in the chip.

The induction of experimental necrotizing enterocolitis (NEC), an intestinal inflammatory disease affecting premature babies, is associated with an impairment of brain development. This is mainly mediated by different intestinal inflammatory cells and factors that migrate and cross the blood-brain barrier to cause brain damage. To use a multi-organ-on-a-chip as a reliable preclinical model, the gut-brain system can emulate the pathologic events occurring in the brain of premature children that have recovered from NEC. Intestinal inflammation will be triggered through the activation of toll-like receptor 4 (TLR4) in the epithelium via lipopolysaccharide (LPS), a main triggering event of NEC. Upon stimulation of TLR4, the inflammatory factors released by the intestinal epithelium will migrate to the brain compartment through the vasculature channels to induce impairment of brain organoids.

The multi-organ-on-chip platforms enable investigation of different types of interactions between organs or other tissues. For example, the multi-organ platform can allow for the evaluation of drug enterohepatic circulation occurring between the small intestine and liver where the drug can be subjected to metabolic transformation. Thus, a drug introduced in the epithelial channel will be absorbed by intestinal epithelial cells and released into the circulation (endothelial cell compartment). The absorbed drug will be then transported to liver organoids in the chip where it can be subjected to metabolic transformation.

Moreover, the incorporation of induced pluripotent stem cells (iPSC) into organ-on-a-chip platforms would allow better testing of physiologic and pathogenic events in the lab, ultimately leading to a more efficient design for future therapeutic approaches at the bedside and allowing a personalized medical approach.

In various embodiments, the disclosed methods of chip fabrication have several advantages over prior PDMS-based chip platforms: they are cost-effective; they are substantially flexible, allowing easy customization based on needs; they include an extracellular matrix mimicking tissue (such as the lamina propria of the intestine), providing the possibility of incorporating different cell types (e.g., immune cells, enteric nervous system cells, and myofibroblasts) found in the different organs, thus providing a higher degree of tissue complexity; they can be adapted to different organs based on anatomy and physiology; they enable multiple organs in one chip allowing for interaction of the organs (“multi-organ-on-a-chip”).

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It is important to note that the construction and arrangement of the devices, assemblies, and steps as shown in the various exemplary embodiments is illustrative only.

Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

### Embodiments

Some sample embodiments are disclosed below, in order to represent illustrative embodiments, which one skilled in the art will understand are may be further modified, combined, constrained, etc. according to the entirety of this disclosure.

Embodiment AA: A microfluidic device for modeling one or more physiological functions of one or more organisms, the microfluidic device comprising a first channel, and a second channel separated from the first channel by an extracellular matrix (ECM) that mimics a connective tissue of the organism.

Embodiment AB: The microfluidic device of Embodiment AA, wherein the ECM comprises a first layer at least partly covering the first channel, and a second layer at least partly covering the second channel.

Embodiment AC: The microfluidic device of either Embodiment AA or AB, the ECM is part of a lamina propria of a selected tissue of an organism.

Embodiment AD: The microfluidic device of any of Embodiments AA to AC, wherein the first channel is in fluid communication with a first set of one or more reservoirs, and the second channel is in fluid communication with a second set of one or more reservoirs.

Embodiment AE: The microfluidic device of any of Embodiments AA to AD, wherein the microfluidic device is an organ-on-a-chip that simulates one or more physiological functions of an organ.

Embodiment AF: The microfluidic device of any of Embodiments AA to AE, further comprising a set of one or more additional channels for modeling interaction between at least two organs.

Embodiment AG: The microfluidic device of any of Embodiments AA to AF, wherein the ECM comprises any combination of: basement membrane matrix, one or more proteins and glycoproteins such as collagens, elastin, laminins and tenascins, proteoglycans and glycosaminoglycans, hyaluronan, polyethylene glycol n-hydroxysuccinimide (PEG-NHS), and/or one or more salts.

Embodiment AH: The microfluidic device of any of Embodiments AA to AG, wherein the ECM comprises: one or more insoluble proteins; collagen; a basement membrane matrix; polyethylene glycol n-hydroxysuccinimide (PEG-NHS); one or more glycoproteins; and a salt.

Embodiment AI: The microfluidic device of either Embodiment AG or AH, wherein the one or more insoluble proteins comprises fibrin, the collagen is type I collagen, and the one or more glycoproteins comprises laminin.

Embodiment AJ: The microfluidic device of any of Embodiments AA to AI, wherein the ECM extends over both the first channel and the second channel.

Embodiment AK: The microfluidic device of any of Embodiments AA to AJ, wherein the ECM comprises a first channel comprising epithelial cells and a second channel comprising endothelial cells.

Embodiment BA: A method of fabricating a microfluidic device, the method comprising: using a sacrificial material to three-dimensional (3D) print a set of channels and a set of reservoirs; covering the set of channels with an extracellular matrix (ECM); and warming the sacrificial material to yield perfusable channels once the set of channels is flushed.

Embodiment BB: The method of Embodiment BA, further comprising rocking the microfluidic device to allow media to go between reservoirs through the set of channels, wherein the rocking provides perfusion of the set of channels.

Embodiment BC: The method of either Embodiment BA or BB, wherein the ECM is part of a lamina propria of an organ or tissue of an organism, and wherein the warming is performed after embedding the set of channels and the lamina propria in a fibrin-PEG-NHS gel.

Embodiment BD: The method of any of Embodiments BA to BC, wherein the sacrificial material comprises a gelatin.

Embodiment CA: A method of fabricating a microfluidic device, the method comprising: printing a set of channels and a set of reservoirs; casting a sacrificial material into the set of channels; embedding the set of channels in an extracellular matrix; and flushing the sacrificial material from the set of channels.

Embodiment CB: The method of Embodiment CA, wherein the set of channels are patterned by casting liquid gelatin into a printed resin mold.

Embodiment CC: The method of either Embodiment CA or CB, further comprising obtaining, through gelation of the resin mold, a gelatin structure that is successively encapsulated in the ECM.

Embodiment CD: The method of any of Embodiments CA to CC, further comprising, after gelation of the ECM, melting and flushing the gelatin structure to leave behind the set of channels separated by the ECM and connected to the set of reservoirs.

Embodiment CE: The method of any of Embodiments CA to CD, wherein the method comprises using digital light processing (DLP) printing or stereolithography (SLA) printing to print the set of channels.

Embodiment CF: The method of any of Embodiments CA to CE, wherein the set of channels are micromesh channels printed with a photocurable polymer resin.

What is claimed:

1. A microfluidic device for modeling one or more physiological functions of one or more organisms, the microfluidic device comprising a first channel, and a second channel separated from the first channel by an extracellular matrix (ECM) that mimics a connective tissue of the organism.

2. The microfluidic device of claim 1, wherein the ECM comprises a first layer at least partly covering the first channel, and a second layer at least partly covering the second channel.

3. The microfluidic device of claim 1, the ECM being part of a lamina propria of a selected tissue of an organism.

4. The microfluidic device of claim 1, wherein the first channel is in fluid communication with a first set of one or more reservoirs, and the second channel is in fluid communication with a second set of one or more reservoirs.

5. The microfluidic device of claim 1, wherein the microfluidic device is an organ-on-a-chip that simulates one or more physiological functions of an organ.

6. The microfluidic device of claim 1, further comprising a set of one or more additional channels for modeling interaction between at least two organs.

7. The microfluidic device of claim 1, wherein the ECM comprises any combination of:
basement membrane matrix, one or more proteins, one or more glycoproteins, polyethylene glycol n-hydroxysuccinimide (PEG-NHS), and/or a salt.

8. The microfluidic device of claim 1, wherein the ECM comprises: one or more insoluble proteins; collagen; a basement membrane matrix; polyethylene glycol n-hydroxysuccinimide (PEG-NHS); one or more glycoproteins; and a salt.

9. The microfluidic device of claim 8, wherein the one or more insoluble proteins comprises fibrin, the collagen is type I collagen, and the one or more glycoproteins comprises laminin.

10. The microfluidic device of claim 1, wherein the ECM extends over both the first channel and the second channel.

11. The microfluidic device of claim 1, wherein the ECM comprises a first channel comprising epithelial cells and a second channel comprising endothelial cells.

12. A method of fabricating a microfluidic device, the method comprising:
using a sacrificial material to three-dimensional (3D) print a set of channels and a set of reservoirs;
covering the set of channels with an extracellular matrix (ECM); and
warming the sacrificial material to yield perfusable channels once the set of channels is flushed.

13. The method of claim 12, further comprising rocking the microfluidic device to allow media to go between reservoirs through the set of channels, wherein the rocking provides perfusion of the set of channels.

14. The method of claim 12, wherein the ECM is part of a lamina propria of an organ of an organism, and wherein the warming is performed after embedding the set of channels and the lamina propria in a fibrin-PEG-NHS gel.

15. A method of fabricating a microfluidic device, the method comprising:
printing a set of channels and a set of reservoirs;
casting a sacrificial material into the set of channels;
embedding the set of channels in an extracellular matrix; and
flushing the sacrificial material from the set of channels.

16. The method of claim 15, the set of channels are patterned by casting liquid gelatin into a printed resin mold.

17. The method of claim 15, further comprising obtaining, through gelation of the resin mold, a gelatin structure that is successively encapsulated in the ECM.

18. The method of claim 15, further comprising, after gelation of the ECM, melting and flushing the gelatin structure to leave behind the set of channels separated by the ECM and connected to the set of reservoirs.

19. The method of claim 15, wherein the set of channels are printed using digital light processing (DLP) printing or stereolithography (SLA) printing.

20. The method of claim 15, wherein the set of channels are micromesh channels printed with a photocurable polymer resin.
